(19) United States
(12) Patent Application Publication
(10) Pub. No.: US 2024/0264146 A1
(43) Pub. Date: Aug. 8, 2024

(54) A DEVICE FOR DETECTING HEALTH DISORDERS FROM BIOLOGICAL SAMPLES AND A DETECTION PROCESS

(71) Applicant: Ricardo Daniel DE SIMONE, Caba (AR)

(72) Inventor: Ricardo Daniel DE SIMONE, Caba (AR)

(21) Appl. No.: 18/566,380

(22) PCT Filed: Jun. 3, 2022
(86) PCT No.: PCT/IB2022/055180

§ 371 Date: Dec. 1, 2023

Related U.S. Application Data
(60) Provisional application No. 63/196,336, filed on Jun. 3, 2021.

(51) Int. Cl. G01N 33/497; G01J 3/443; G01N 1/44; G01N 21/67; G06V 10/774; G06V 20/69

(52) U.S. Cl. CPC G01N 33/4975; G01J 3/443; G01N 1/44; G01N 21/67; G06V 10/774; G06V 20/69

(57) ABSTRACT

The present invention relates to instruments and processes for detecting compounds in gas samples. In particular, for detecting health disorders from biological samples, more preferably for detecting diseases from breath samples of a mammal. It is included among the methods and instruments for the diagnosis of COVID-19.

### FIELD OF THE INVENTION

The present invention relates to instruments and processes for detecting compounds in gas samples. In particular, for detecting health disorders from biological samples, more preferably for detecting diseases from breath samples of a mammal. It is included among the methods and instruments for the diagnosis of COVID-19.

### STATE OF THE ART

Human respiration contains a significant amount of volatile organic compounds (VOCs) that are the product of metabolic activity. These VOCs can differ according to genetic or environmental factors such as age, weight, sex, lifestyle or eating habits, and can influence the chemical composition of the breath of a person, depending on the amount and concentration of these compounds. Diseases can also cause an alteration of VOCs in exhaled breath during respiration.

This relationship between smells present in the breath and diseases has been known to physicians for several hundred years. The detection of diseases through smell dates back to the fourth century, when the physician, based on experience, was able to determine what disease a person suffered from the smell of their breath. For example, a fruity-smelling breath was identified as a sign of ketoacidosis associated with diabetes, and the odor of ammonia may be related to kidney failure. This method was not accurate, as the disease had to be in an advanced stage to be detected by human olfaction. However, technological advancement has allowed, on this basis, the development of electronic systems to diagnose diseases through respiration and provide information on the state of the human body.

Electronic noses represent an innovative method of VOC sampling because these devices allow online recognition of complex mixtures of VOCs using nanosensor arrays in combination with learning algorithms. Each set of sensors is sensitive to different fractions of the VOCs mixture, and the arrays exhibit good discrimination performance in combination with high sensitivity and short response time.

Therefore, the use of electronic noses constitutes an attractive alternative to the standard and preferred method that is currently used for the diagnosis of COVID-19, which is a real-time reverse transcription-polymerase chain reaction (RT-PCR) based on a nasopharyngeal and/or oropharyngeal swab. In this sense, their speed in diagnosis, their low cost and the fact that they do not require an invasive sampling procedure are the main advantages of electronic noses.

Among the devices for diagnosing diseases from VOCs in breath samples that can be found in the state of the art, it can be mentioned a device for the diagnosis of pneumonia using exhaled breath that is disclosed in document US20190167152 A1. Said device consists of a breath detector operable to capture and hold a volatile organic compound (VOC) that is contained within an exhaled breath; a breath VOC analyzer in communication with the breath detector apparatus, where said VOC is 1-propanol and where said breath VOC analyzer comprises an electronic nose configured to determine the VOC of interest or a gas chromatography-mass spectrometry (GC-MS) analyzer. On the other hand, document WO2020/160753 A1 introduces an apparatus to reduce the impact of confounding factors for real-time analysis of the chemical composition of respiration, where said apparatus is coupled with a chemical analyzer. The analyzer comprises an ionizer that produces ions by ionizing the molecules of interest from said flow passed to the analyzer, and an ion analyzer that analyzes said ions. Said ion analyzer can be a mass spectrometer, an ion mobility spectrometer, or a combination of a mass spectrometer and an ion mobility spectrometer. In addition, the document https://doi.org/10.1007/s00464-020-08169-0 discloses the use of a device called “Aconose” from “The Aeonose Company” for the diagnosis of COVID-19 disease. This device contains three metal oxide type sensors that detect CO, N₂ and VOCs. At each measurement, the sensors go through a temperature cycle. The exhaled VOCs react via redox reactions with the sensors, inducing changes in conductivity and generating numerical patterns that are later stored and analyzed.

The breath analysis devices for disease detection available on the market are based on mass spectrometers, ion mobility spectrometers, gas chromatography, or combinations of them, which implies high costs and large sizes. The different types of sensors have certain disadvantages such as slow recovery (metal oxide semiconductor), drift in response (metal oxide semiconductor, conductive polymer, surface acoustic wave), low noise immunity (photoionization detector) and lack of reproducibility between sensors of different sets (conductive polymer, metal oxide semiconductor field effect, quartz crystal microbalance, surface acoustic wave) and of the same sensor in the long term. If these disadvantages are to be avoided, the alternative would be to use arrays of different types of sensors to overcome these limitations and maximize their advantages. However, this implies higher costs and more complex devices.

On the other hand, the document doi.org/10.1039/c6ja00226a, introduced a system based on corona discharge induced plasma spectroscopy. This system was employed to determine the concentration of oxygen and nitrogen mixtures. Corona Discharge Induced Plasma Spectroscopy (CDIPS) technique is based on generating plasma from an electrical corona discharge across a constant gas flow. The radiation from the plasma provides information about its physical properties such as composition, electron density and temperature. Thus, the behavior of the emitter species can be correlated with changes in the density of ions and the electron temperature allowing the quantification of such species. The emission from the plasma is collected by a UV-Visible spectrometer obtaining a spectrum consisting of discrete and narrow atomic emission lines and wider bands of molecular species such as N₂ or O₂, characteristic of the elements present in the plasma. The system showed high reproducibility and could be used in different laboratories, obtaining the same information in each laboratory. This technology needs a very expensive standard spectrometer.

There is still a need for a device that allows diagnosing diseases such as COVID-19 by adapting the technology of corona discharge induced plasma spectrometry to be able to detect changes in the composition of exhaled breath.

There is still a need for a diagnosis that could be made in a safe, quick, economical and non-invasive way; in which the possibility of contamination and depletion of the chemical sensors traditionally used in electronic noses is eliminated.

### BRIEF DESCRIPTION OF THE FIGURES

FIG. 1. Disease detection device from breath samples.

FIG. 2. Anode and cathode cross-section and corona discharge plasma representation.

FIG. 3. Image processing scheme.

FIG. 4. Processing scheme for the image data of the device of the present invention.

FIGS. 5 and 6 are photographs of one embodiment of the device of the present invention.

### BRIEF DESCRIPTION OF THE INVENTION

The present invention provides a device for detecting health disorders from biological samples, comprising means for generating an electric discharge in said samples; at least one optical sensor and means for images processing. Said device does not require sophisticated detection systems, but also can perform analyses with high efficiency and very low cost, since it only requires an image detector to make its diagnosis.

The present invention provides a device that subjects said biological samples, preferably gaseous samples, more preferably breath samples, to an electrical discharge that is able to induce plasma.

Wherein said health disorders comprise diseases.

The present invention provides an image sensor and means for process the images by artificial intelligence. Said electrical discharge could be a corona discharge. Therefore, this system could be named as a corona discharge induced plasma digital spectroscopy.

This system has a high reproducibility for diagnosing COVID-19. This technology could offer the possibility of diagnosing or screening health disorders in a non-invasive and rapid way with low-cost instruments.

Wherein said disease or health disorder preferably is selected from the group consisting of a viral infection, a bacterial infection, breast cancer, prostate cancer, lung cancer, colon cancer presence of alcohol in blood, diabetes, kidney failure or presence of cannabinoids in blood. More preferably is COVID-19 disease. The bacterial infection may be pneumonia. Also, may be any health disorder that can manifest itself through biomarkers present in exhaled breath.

The device for detecting compounds, according to the present invention, can also detect volatile compounds presents in a biological sample selected from the group consisting of urine sample and stool sample.

In a preferred embodiment, the present invention comprises: a sample inlet, a carrier gas inlet, a homogenization sector and a gas outlet (1); at least one ionization chamber (2); at least one optical sensor (3); and an image storage and processing system (4). In a more preferred embodiment of the invention, said sample inlet, said carrier gas inlet, said homogenization sector and said gas outlet comprise: a sample inlet port; a sample inlet duct that communicates said sample inlet port with the ionization chamber; a gas outlet port; a gas outlet duct that communicates the ionization chamber with said gas outlet port; an ionization product retention filter; a sample inlet and outlet pump; a low voltage power supply powering said pump; a carrier gas inlet port; a carrier gas inlet duct; an element that links the sample and carrier gas inlet ducts; at least one sample flow control valve; at least one carrier gas flow control valve. Wherein said ionization chamber comprises, preferably, a body with two electrodes: anode and cathode; a high or low voltage power supply, powering said electrodes. And wherein said ionization chamber, preferably, also comprises an optical fiber that links the inside of said ionization chamber (2) with the optical sensor (3).

In a preferred embodiment of the invention, said anode comprises a central electrode and said cathode is a cylinder forming a coaxial needle-cylinder geometry; and said optical sensor comprises at least one microscope or a photographic camera, that could be a mobile telephone camera.

In another preferred embodiment of the invention, said image storage and processing system (4) comprises: a computer connected to said optical sensor (3), which receives, stores and analyzes, by artificial intelligence, the images of plasma produced by the samples when passing through the electric arc between the electrodes.

In another preferred embodiment, the invention comprises a PC USB connection; a lithium-ion battery; an integrated touch screen; the necessary elements to establish a wireless connection.

Wherein the device of the invention for detecting diseases from breath samples comprises an electric discharge induced plasma digital spectrometer.

In another embodiment of the invention said device further comprises a sample reservoir wherein solid or liquid samples are introduced; which comprises means to vaporize liquid or solid samples. Wherein said solid or liquid samples could be stool and urine. In this embodiment said means to vaporize said liquid or solid samples could comprise a Laser.

Another object of the present invention is a process for detecting diseases from breath samples that, preferably, uses the device of the present invention and comprises the following steps:
a) providing a container with a breath sample;
b) providing a carrier gas that mixes with the breath sample to carry said sample into an ionization chamber in a homogeneous and controlled manner;
c) Ionizing said carrier gas and said breath by means of an electric arc;
d) capturing and storing images of the plasma generated in said electric arc;
e) evacuating the ionization chamber by circulating said pure carrier gas, in absence of a breath sample;
f) processing said images by artificial intelligence to determine if said images are compatible with breath samples from sick people;
g) giving a visual indication of the result.

Another object of the present invention is a process for detecting diseases from breath samples that comprises the following steps:
a) providing a carrier gas that mixes with the sample to carry said sample into an electric arc;
b) Ionizing said carrier gas and said sample by means of an electric arc;
c) capturing d storing images of the plasma generated in said electric arc;

Said process, could also comprise the steps of:
d) processing said images by artificial intelligence to determine if said images are compatible with established parameters;
e) giving a visual indication of the result.

Another object of present invention is a container comprising flexible material evacuated and sterilized with only one gas entrance that could be filled with the exhales of breath as biological sample of the present invention.

Another object of present invention is an image analysis process called digital spectroscopy that utilizes the device of the present invention comprising the following steps:
a. generation of the database for the image training that represents the wavelengths to generate the spectra;
b. generation of the spectrum of each training image, fitting of the data and cross validation of the model;
c. generation of spectra of the samples to be analyzed and their prediction.

### DETAILED DESCRIPTION OF THE INVENTION

The device for detecting compounds, diseases or health disorders from gaseous samples by electric discharge induced plasma digital spectroscopy, an object of the present invention, is characterized for being capable of detecting variations in the concentrations of volatile organic compounds (VOCs) that are present in the breath, which act as disease-related biomarkers. According to the present invention, the device is capable of generating plasma from an electrical discharge. When the breath samples pass through the electric arc generated by the electric discharge, they are ionized. Therefore, changes are produced in the density of ions and the temperature of the electrons, allowing the quantification of these species. In this way, a set of optical sensors can register these changes and, through image processing and analysis (called digital spectroscopy) along with neural network training, the device is capable of detecting diseases.

Health disorder in this document comprises viral infections, viral infection of COVID-19, viral infection of pneumonia, diabetes, bacterial infections, bacterial infection of pneumonia, kidney failure, breast cancer, prostate cancer, lung cancer and colon cancer and any disorder or disease that can manifest itself through biomarkers present in exhaled breath. Also, health disorder in this document comprises alterations to the good health caused by the presence of drugs or substances like alcohol or cannabinoids in blood.

The device for detecting diseases from breath of the present invention comprises a sample inlet, a carrier gas inlet, a homogenization sector and a gas outlet (1); at least one ionization chamber (2); at least one optical sensor (3); and an image storage and processing system (4).

In a preferred embodiment, said sample inlet, said carrier gas inlet, said homogenization sector and said gas outlet (1); said ionization chamber (2); said optical sensor (3); and said image storage and processing system (4) are included in the same housing.

Wherein said sample inlet; said carrier gas inlet, said homogenization sector and said gas outlet (1) comprise a sample inlet port; a sample inlet duct that communicates said inlet port with the ionization chamber; a gas outlet port; a gas outlet duct that communicates the ionization chamber with said outlet port; an ionization products retention filter; a sample inlet and outlet pump by means of which the flow rate and working pressure of the system are regulated; a low voltage power supply powering said pump; a carrier gas inlet port; a carrier gas inlet duct; an element that links the sample and carrier gas inlet ducts; at least one sample flow control valve and at least one carrier gas flow control valve.

In a preferred embodiment of the present invention, the carrier gas used is nitrogen, which can be generated “in situ” in a membrane separation system from where nitrogen is obtained from air. In another embodiment, said nitrogen source may consist of a nitrogen cylinder.

In another embodiment of the present invention, the carrier gas can be air, where the air source can comprise a compressed air cylinder, a synthetic air cylinder, or an ambient air pumping system integrated into the device.

In another embodiment of the present invention, the carrier gas can be any of the group of noble or inert gases.

In another embodiment, the device does not require the use of carrier gas, the gaseous sample it is simply blown over a “grill” located in an external surface of the housing that admits said sample. This design allows the device to be portable, having a size comparable to a smartphone.

In a preferred embodiment of the invention, the element that links the sample and carrier gas inlet ducts is a three-way valve that is placed in the sample inlet duct and allows only the sample to enter the ionization chamber, or only the carrier gas or a mixture of both. Additionally, the sample inlet and carrier gas inlet ducts can comprise flow control valves that allow the control of the flow rate of sample and carrier independently, prior to mixing of sample and carrier gas in the three-way valve.

In another embodiment of the present invention, the element that links the sample and carrier gas inlet ducts can be a T-type or Y-type union.

In an even more preferred embodiment, said flow control valves are automatically operated through software, as needed in the different stages of the breath sample analysis procedure.

In a preferred embodiment, said ionization products retention filter is located inside the gas outlet duct that communicates the ionization chamber with the outlet port. Said filter can be removed to be discarded and replaced by another one after retaining or absorbing the samples that leave the ionization chamber.

In a preferred embodiment, said ionization chamber (2) consists of a body, which comprises two electrodes, anode and cathode. An electric potential difference is applied between said electrodes so that a electric arc (corona discharge) is generated that is capable of ionizing the carrier gas or a mixture of sample and carrier gas that enters said ionization chamber.

Furthermore, said ionization chamber (2) comprises, preferably, a high or low voltage power supply to power said electrodes; and one or a plurality of optical fibers that conduct the light produced by the corona discharge from inside the body of the ionization chamber to the microscope and/or photographic camera. In a preferred embodiment of the invention, the body of said ionization chamber is characterized by having a cylindrical geometry. Furthermore, according to this embodiment, the electrodes are characterized in that the anode consists of a central needle and the cathode is a cylinder forming a coaxial needle-cylinder geometry.

In an embodiment of the present invention, said optical sensor (3) can comprise at least one microscope, or at least one photographic camera of the type used in smartphones.

On the other hand, the image storage and processing system (4) comprises a computer connected to said optical sensor (3), which receives, stores and analyzes by artificial intelligence the plasma images produced by the samples when passing through the electric arc between said electrodes.

Furthermore, the device comprises a PC USB connection; a lithium-ion battery that provides electrical energy to all the components of the device that require electricity for its operation; an integrated touch screen to control the operation of the device; and the necessary elements to establish a WiFi-type connection, in order to be able to transmit the images and diagnostic results to a cloud, and/or bluetooth connection, so that it can be controlled from a smartphone through an “ad hoc” application.

In a preferred embodiment of the invention, an evacuated and sterilized container that has been filled with the exhales of breath is used to collect the breath samples. In this way, direct contact between the user and the device is avoided. Subsequently, said container is connected to the device and the sample is pumped and mixed with the carrier gas in the element that links the sample and carrier gas inlet ducts and then enters the ionization chamber. In an even more preferred embodiment of the invention, said sampling container has a volume greater than 500 mL. In an even more preferred embodiment, the container volume is close to 2000 mL. This volume is considerably greater than that exhaled by an average person (approximately 500 mL), requiring at least cuatro exhalations to complete the filling of the container. Consequently, the products of the beginning, middle and end of an exhalation are collected at the same time as it is also possible to neutralize the differences in pressure and volume of breath delivered by the different users (children, youth, adults and older adults; men and women). In addition, the use of this sampling container considerably reduces the possibility of errors in the reading due to the entry of saliva or unwanted substances.

In another embodiment of the present invention, the device further comprises a sample reservoir wherein solid or liquid samples, such as stool and urine, are introduced. According to this embodiment, the device comprises means to vaporize liquid or solid samples. Preferably, said means to vaporize said liquid or solid samples comprises a Laser. In addition, the device also comprises means to collect said vaporized sample and introduce it into the ionization chamber.

The process of detecting diseases from breath using, preferably, the previously described disease detection device, comprises the following steps:
a) providing a container with a breath sample;
b) providing a carrier gas that mixes with the breath sample to carry said sample into an ionization chamber in a homogeneous and controlled manner;
c) Ionizing said carrier gas and said breath by means of an electric arc;
d) capturing and storing images of the plasma generated in said electric arc;
e) evacuating the ionization chamber by circulating said pure carrier gas, in absence of a breath sample;
f) processing said images by artificial intelligence to determine if said images are compatible with breath samples from sick people;
g) giving a visual indication of the result.

### APPLICATION EXAMPLES

### Example 1

A device of the present invention is described in detail below.

Said device comprises a sample inlet, a carrier gas inlet, a homogenization sector and a gas outlet (1); at least one ionization chamber (2); optical sensor (3); and an image storage and processing system (4). In turn, all these elements are included in the same housing.

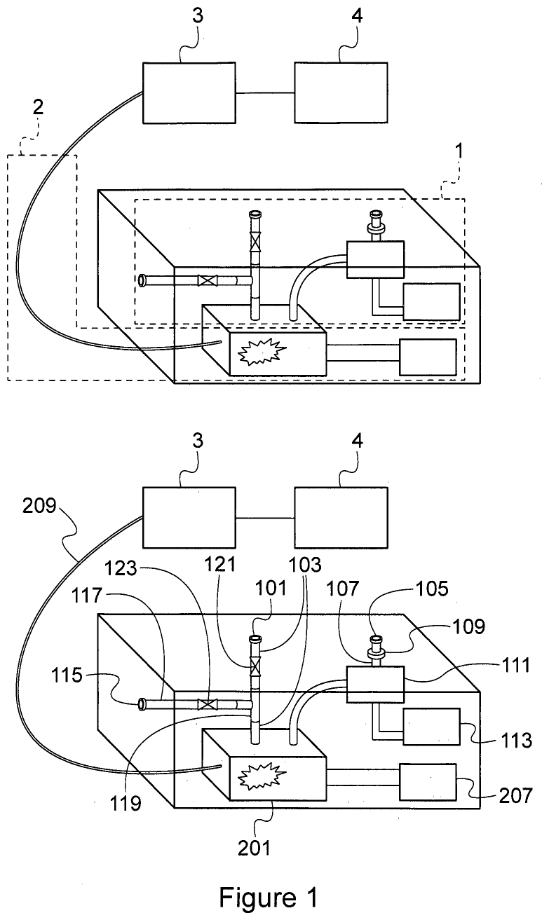

As shown in the diagram of FIG. 1, said sample inlet, said carrier gas inlet, said homogenization sector and said gas outlet (1) comprise a sample inlet port (101); a sample inlet duct (103) communicating said inlet port with the ionization chamber; a gas outlet port (105); a gas outlet duct (107) communicating the ionization chamber with said outlet port; an ionization product retention filter (109); a sample inlet and outlet pump (111); a low voltage power supply powering said pump (113); a carrier gas inlet port (115); a carrier gas inlet duct (117); three-way valve (119) that links the sample and carrier gas inlet duct; a sample flow control valve (121) and a carrier gas flow control valve (123).

Wherein, said flow control valves (121) and (123) are automatically operated through software, as needed in the different stages of the breath sample analysis procedure.

The carrier gas utilized is nitrogen.

Wherein said ionization product retention filter (109) is located inside the gas outlet duct that communicates the ionization chamber with the gas outlet port. Said filter (109) can be removed to be discarded and replaced by another one after retaining or absorbing the samples that leave the ionization chamber.

The ionization chamber (2) consists of a body (201), which comprises two electrodes, anode (203) and cathode (205). In addition, said ionization chamber comprises a high voltage power supply (207). By means of said power supply (207), an electric potential difference is applied to said electrodes so that an electric arc is generated in corona discharge, that is capable of ionizing the carrier gas or mixture of sample and carrier gas that enters the ionization chamber. Said ionization chamber also comprises an optical fiber (209) that links the body (201) of said chamber with the optical sensor (3).

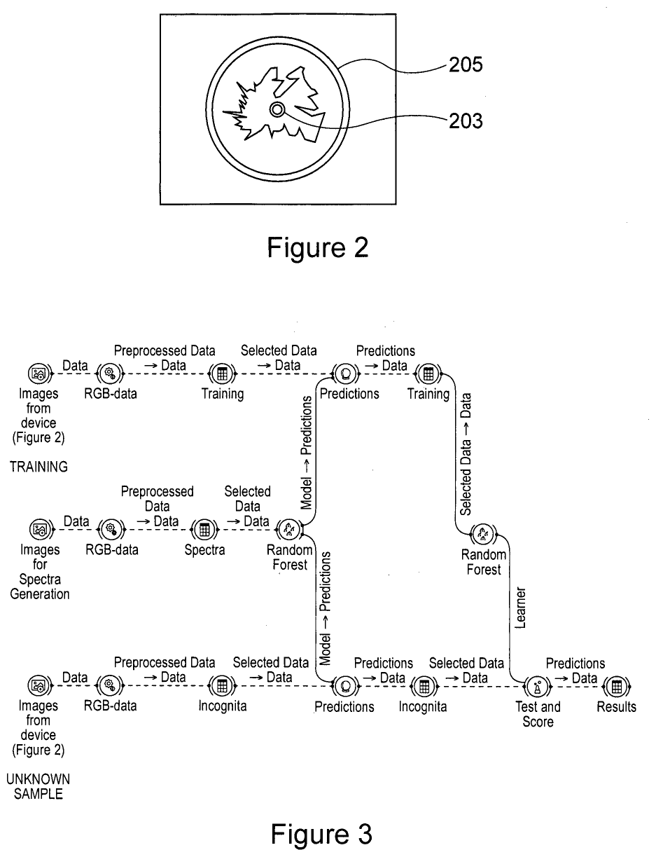

The body of said ionization chamber has a cylindrical geometry and the electrodes are the anode (203) that consists of a central needle and the cathode that is a cylinder (205) forming a coaxial needle-cylinder geometry, as illustrated in FIG. 2.

The optical sensor (3) is a microscope that captures the images of the corona discharge that are generated between the electrodes inside the body of the ionization chamber. The image storage and processing system (4) comprises a computer connected to said microscope (3), which receives, stores and analyzes by artificial intelligence the plasma images produced by the samples when passing through the electric arc between said electrodes.

Furthermore, the device comprises a PC USB connection; a lithium-ion battery that provides electrical energy to all the components of the device that require electricity for its operation, providing an autonomy of at least 3 hours; an integrated touch screen; and the necessary elements to establish wireless connections (WiFi and bluetooth type).

The breath sample container used in this embodiment consists of a previously evacuated and sterilized plastic bag with a volume of 2000 mL.

### Example 2

Collection of samples from volunteers with and without COVID-19 for the assays performed herein and in the following examples.

The samples were taken by means of an inspiratory capacity maneuver followed by the exhalation of a vital capacity volume. The exhaled breath was collected in a sterile 2 L urine drainage bag with a bottom outlet and 120 cm Tube A4 to homogenize the gas mixture. Each sampling bag was properly labeled and isolated in a nylon bag for further analysis. It was decided not to use filters in order to collect all possible markers of the COVID-19 disease.

Each volunteer was instructed to be on a 2-hour fast prior to providing breath samples. The volunteer was also requested to inflate the bag through the outlet tube and was asked to avoid blowing saliva.

### Breath Sample Collection Procedure:

The volunteer was asked to inhale deeply and fully (in order to reach full lung capacity) and then to exhale as hard and fast as possible. The volunteer was encouraged to do it with enough strength, keeping the nose covered, to complete the established volume and fill the plastic container. The typical exhalation is estimated to be 500 mL, therefore at least four exhalations were required. Once the sampling bag was full, the valve was closed and stored in a security bag with its label.

### Example 3

### Chemometric Analysis and Artificial Intelligence.

To perform the identification of the samples and determine the discrimination capability of the device, the following procedure was carried out to train and identify the analyzed samples. These samples correspond to volunteers previously diagnosed with or without COVID-19 disease.

The image analysis process called digital spectroscopy is divided into three steps:
a. Generation of the database for the image training that represents the wavelengths to generate the spectra.
b. Generation of the spectrum of each training image, fitting of the data and cross validation of the model.
c. Generation of spectra of the samples to be analyzed and their prediction.

Note: all the utilized Machine Learning (ML) models belong to the “scikit-learn” python library

To develop the model that generates the spectra of the images, images that represent designated “wavelengths” were used. These are the variables of the image training model. The procedure to generate the models is similar to what is done with the images to be studied: The “PIL” library is used to take the RGB composition of each pixel in the image. The corresponding arbitrary length is assigned to each of these combinations of values. Once the dataframe with all the combinations has been generated, a Machine Learning (Random Forest) model is generated. This model (from now on called pixelMod) is later used to define which is the length that belongs to each pixel of the studied images.

The device takes images (and records a video with them) of the corona discharge, similar to the one shown in FIG. 2. These images are acquired through the “CV2” library.

To develop the training model, the same PIL library is used to extract the information of the pixels of each image to be analyzed, excluding the black pixels. Once this data has been extracted, the pixelMod is used to determine to which length each pixel belongs. After the assignment is completed, the table is generated with all the pixels of each length that each of the images has. This table is the new basis for generating a ML (Random Forest) training model. This classification model will have two variables: POSITIVE (COVID-19)—NEGATIVE. The gridsearch_cv (cross validation) was used to iterate between different parameters and then obtain the optimal one. Cross validation was also used to check which was the best score obtained. In the event that the expected threshold were exceeded, it then proceeds with the last step. This model (from now on called entrenaMod) is used to predict which group the unknown measurements correspond to.

In the case of the samples to be analyzed, the procedure is similar as in the training step: the spectra table of each image is generated with the pixelMod and once the table is completed, it is predicted with the entrenaMod. This result will be exposed in a probabilistic way for each variable, and the resulting numbers are the average image probabilities of the measurement. The processing scheme is detailed in FIG. 3.

### Example 4

The volunteers were asked to fill a form with relevant data in order to control the analysis protocol. The requested data was: age, gender and previous illnesses or conditions such as chronic obstructive pulmonary disease (COPD), if they are smokers or not, asthma, diabetes and high blood pressure e (HBP). The collected data is shown in Table 1.

TABLE 1
Volunteers data.

| | | | Previous | |
|---|---|---|---|---|
| | | | illnesses (COPD, | |
| Name/ | | | HBP, smoker, | Volunteer |
| Identifier | Age | Gender | asthma, diabetes) | code |
| M1 | 27 | Female | — | AM1 |
| M2 | 85 | Male | COPD, HBP | AM2 |
| M3 | 91 | Female | — | AM3 |
| M4 | 52 | Female | HBP | AM4 |
| M5 | 57 | Male | asthma, HPB | AM5 |
| M6 | 59 | Female | HBP | AM6 |
| M7 | 55 | Female | — | CM1 |
| M8 | 27 | Male | — | CM2 |
| M9 | 62 | Male | diabetes | CM3 |
| M10 | 55 | Male | HBP | CM4 |

The samples were labeled and classified into two groups according to the presence or absence of the COVID-19 disease, determined by PCR analysis. The classification of the samples is shown in Table 2.

TABLE 2
Identification of samples for data processing

| PCR analyses | Symptoms | Group | Volunteer code |
|---|---|---|---|
| Positive (+) | More than one | PS + N1 | AM1 |
| | | | AM2 |
| | | | AM3 |
| | | | AM4 |
| | | | AM5 |
| | | | AM6 |
| Negative (−) | More than one | PN − NS2 | CM1 |
| | or without | | CM2 |
| | | | CM3 |
| | | | CM4 |

The set of samples that were used to train the model is not included in the prediction of results. From each container/sample five measurements were performed and two of these measurements were used for training. On the other hand, the selection criterion in the model was to classify the scores greater than 0.45 as “DOUBTFUL”, greater than or equal to 0.55 as “POSITIVE” and less than 0.45 as “NEGATIVE”. Considering Table and the prediction percentage (Positive/Negative/Doubtful) according to the analysis by digital spectroscopy, the following results shown in Table 3 were obtained.

TABLE 3
COVID-19 diagnosis of the group of volunteers according
to the device of the present invention.

| PCR | | | Volunteer | Positive | Negative | Doubtful |
|---|---|---|---|---|---|---|
| analyses | Symptoms | Group | code | (%) | (%) | (%) |
| Positive | More than | PS + N1 | AM1 | 100 | 0 | 0 |
| (+) | one | | AM2 | 58.8 | 21.2 | 20 |
| | | | AM3 | 61.0 | 23.7 | 15.3 |
| | | | AM4 | 74.8 | 16.7 | 8.5 |
| | | | AM5 | 88.9 | 5.5 | 5.6 |
| | | | AM6 | 76.4 | 12.4 | 11.2 |
| Negative | More than | PN − NS2 | CM1 | 1.3 | 98.0 | 2.7 |
| (−) | one | | CM2 | 1.3 | 96.1 | 2.7 |
| | or without | | CM3 | 25.3 | 55.6 | 19.1 |
| | | | CM4 | 3.4 | 85.6 | 11.0 |

From these results, it can be observed that a healthy volunteer is reported with a probability close to 90%, while a healthy volunteer who has diabetes gives a value of 55% that is borderline. This shows the power of the present invention to detect diseases other than COVID-19 in a volunteer.

In Positive cases, it can be observed that the percentage of Doubtful and Negative are very similar and of the same order, which would indicate a prevalence of the Positive case. These results are validated by PCR analysis, which shows not only a high correspondence with other types of analysis, but also that it can be used as a method of in situ detection of COVID-19.

To determine the precision of the measurement system, all the images of the positive and negative cases of COVID-19, a total of 4652, were used. With this information, Table 4 was constructed.

TABLE 4
Summary of the results from image analysis.

| | Positive (%) | Negative (%) | Doubtful (%) | |
|---|---|---|---|---|
| | (prediction) | (prediction) | (prediction) | Total |
| Positive | 87.9 | 4.9 | 7.1 | 3036 |
| (real) | | | | |
| Negative | 77.4 | 80.1 | 8.5 | 1616 |
| (real) | | | | |

From Table 4 it can be observed that the device of the present invention allows the determination of the COVID-19 disease in the exhaled breath of volunteers, in different phases of the disease. The system has a reliability close to 95% with a 5% false negative.

### Example 5

### Chemometric Analysis and Artificial Intelligence.

To identify the samples and determine the discrimination potential of the device of the present invention, the following procedure was conducted.

The report on the interpretation of diagnostic tests for SARS-CoV-2 (Spanish Society of Infectious Diseases and Clinical Microbiology, Instituto de Salud Carlos III) was considered for the training of the device. Table 5 shows the different stages of the disease according to the tests performed by the methods indicated.

TABLE 5
Summary of general interpretation for the respective tests.

| PCR | Ag | IgM | IgG | Interpretation |
|---|---|---|---|---|
| + | − | − | − | Pre-symotomatic phase |
| + | +/− | +/− | +/− | Initial phase |
| +/− | − | + | +/− | 2^{nd} Phase (8 to 14 days) |
| +/− | − | ++ | ++ | 3^{rd} Phase >15 days |
| + | − | +/− | ++ | Past infection, immune |

According to this table the method developed can detect the disease from the initial phase to the third phase. To determine whether the method detects COVID-19 disease and is not confused with other pre-existing respiratory diseases, each volunteer was specifically required to indicate whether he or she had any pulmonary disease prior to performing this voluntary test. Table 6 shows the data requested from each volunteer and the results of the tests that were performed in simultaneous with exhaled breath sampling.

TABLE 6
Control for volunteer testing.

| | | | Previous Disease | |
|---|---|---|---|---|
| Sample/ | | Sex | (COPD, Smoker, | PCR/Ag |
| Code | Age | (M/F) | asthma, diabetes) | (P/N) |
| M000 | 65 | M | D | N |
| M001 | 78 | M | | N |
| M002 | 75 | F | D | N |
| M003 | 51 | F | | N |
| M004 | 46 | F | | N |
| M005 | 43 | F | | P |
| M006 | 51 | M | | P |
| M007 | 20 | F | | P |
| . . . | . . . | . . . | . . . | . . . |
| . . . | . . . | . . . | . . . | . . . |
| . . . | . . . | . . . | . . . | . . . |
| M032 | 39 | F | | P |
| M033 | 34 | F | | N |
| M034 | 34 | M | | N |
| M035 | 60 | M | | P |
| M036 | 40 | M | D | N |
| M037 | 26 | M | | — |
| M038 | 25 | M | S | — |
| M039 | 36 | F | S | — |
| M040 | 22 | M | | — |
| M041 | 51 | F | S/C | P |
| M042 | 46 | F | S/A | P |
| M043 | 16 | F | | N |
| M044 | 57 | F | S/C | — |
| M045 | 65 | M | D | — |
| M046 | 55 | M | | — |

The samples were separated into two sets, with or without COVID-19 disease, to train the device. Table 6 shows the identification of each sample for training purposes. The identification was conducted considering the results obtained by PCR and/or Abbott test.

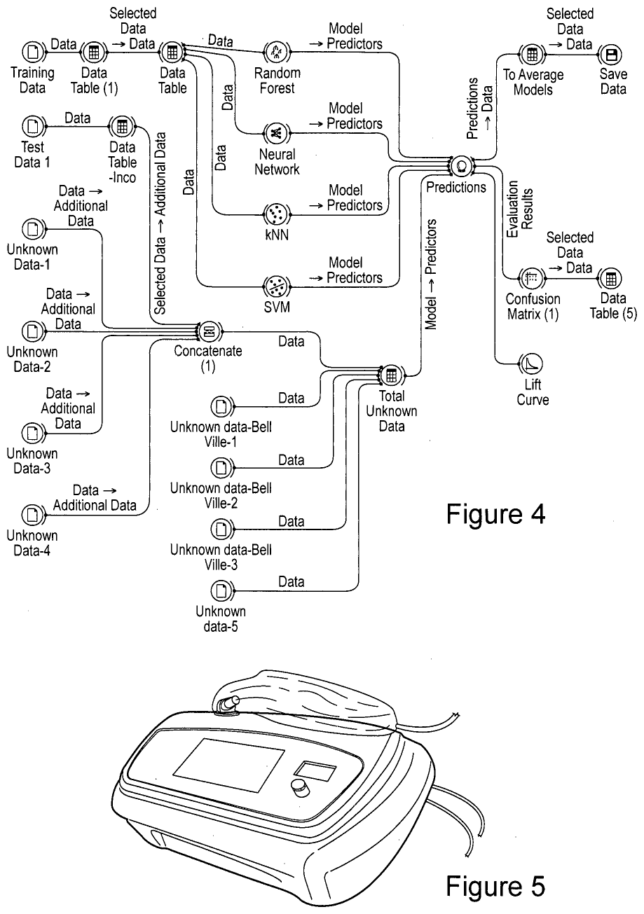

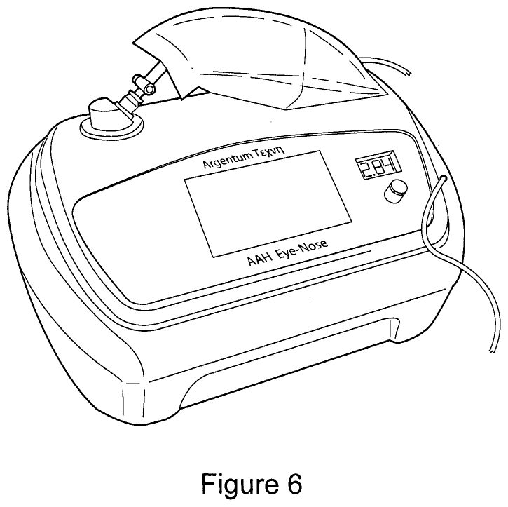

The FIG. 4 shows an example of data reading, processing, classification, cross-validation, and presentation of results. The map is composed with different block with widgets: File, Data Table, Pre-process Spectra and Spectra Visualization.

Another block is composed with different classifiers, block of evaluations with Test and Score and Confusion Matrix. Finally, block of Graphic Results and Python scripts. The images were analyzed using the Pillow Phyton library. This library calculates and returns the entropy for the image. A bilevel image (mode “1”) is treated as a greyscale (“L”) image by this method. If a mask is provided, the method employs the histogram for those parts of the image where the mask image is non-zero. The mask image must have the same size as the image, and be either a bi-level image (mode “1”) or a greyscale image (“L”). Using the same library the Hue-Saturation-Value (HSV) functions, given as hsv (hue, saturation %, value %) where hue and saturation are the same as HSL, and value is between 0% and 100% (black=0%, normal=100%). For example, hsv (0, 100%, 100%) is pure red. This format is also known as Hue-Saturation-Brightness (HSB), and can be given as hsb (hue, saturation %, brightness?), where each of the values are used as they are in HSV. This information is then used to train the different chemometric models (neural networks, random forest, etc.), as shown in FIG. 4.

To evaluate specificity and sensitivity, 28 measurements were taken that were not used as a data training. The results obtained by the different AI methods were then averaged and those exceeding 0.55 probability were assigned as positive and negative. As an example, if the average of (Neural Networks, Random Forest, k-nearest neighbors and linear support vector machines) gives 0.55 for positive, the sample is considered COVID-Positive and if the average of (Neural Networks, Random Forest, k-nearest neighbors and support vector machines) gives 0.55 for negative the sample is considered COVID-Undetectable. On the other hand, the average of the probabilities that resulted between 0.45 and 0.55 were considered as doubtful and were not used to determine the sensitivity (true positive rate (TPR)) and sensitivity (true negative rate (TNR)). The results are shown in table 7.

TABLE 7
Number of samples analyzed by PCR and the device
of example 1. PCR was assumed as a Gold Standard.

| Positive | Positive | Negative | Negative | | |
|---|---|---|---|---|---|
| PCR | Device | PCR | Device | TPR | TNR |
| 10 | 7 | 18 | 15 | 91% | 82% |

Using the data in Table 7 it is also possible to obtain Positive Predictive Value (PPV) and Negative Predictive Value (NPV) indicating the prevalence of COVID-19 in the sampled population using PCR as the Gold Standard. In this case a PPV of 70% and a NPV of 80% were determined, which shows that the analysis system can be used as a screening method.

It has been shown that the system allows to detect with an accuracy close to 91% and it can report false negatives in values less than 5%. On the other hand, the system used requires very low-cost disposables and the time to process a sample or a bag containing exhaled air is in the order of 3 minutes. In addition, the method used is not invasive for the user so it is not bothersome, and the sampling time is relatively low. This analysis system shows that it can be used as a screening method to test in very short times, large populations, for the monitoring of the COVID-19 disease.

1. A device for detecting health disorders from biological samples, comprising means for generating electric discharge in said samples; at least one optical sensor and means for images processing.

2. The device of claim 1 wherein said electric discharge generates plasma in said samples and said samples are gaseous samples.

3. The device of claim 1 wherein said health disorders comprise diseases.

4. The device of claim 1 wherein said biological samples comprise breath samples.

5. The device of claim 1 comprising: a sample inlet, a carrier gas inlet, a homogenization sector and a gas outlet (1); at least one ionization chamber (2); at least one optical sensor (3); and an image storage and processing system (4).

6. The device of claim 5 wherein said sample inlet, said carrier gas inlet, said homogenization sector and said gas outlet comprise: a sample inlet port; a sample inlet duct that communicates said inlet port with the ionization chamber; a gas outlet port; a gas outlet duct that communicates the ionization chamber with said outlet port; an ionization product retention filter; a sample inlet and outlet pump; a low voltage power supply powering said pump; a carrier gas inlet port; a carrier gas inlet duct; an element that links the sample and carrier gas inlet ducts; at least one sample flow control valve; at least one carrier gas flow control valve.

7. The device of claim 5, wherein said ionization chamber comprises a body with two electrodes: anode and cathode; a voltage power supply, powering said electrodes.

8. The device of claim 7, wherein said power supply comprises a high voltage power supply.

9. The device of claim 7, wherein said power supply comprises a low voltage power supply.

10. The device of claim 5, wherein said ionization chamber further comprises an optical fiber that links the interior of the body of said ionization chamber (2) with said optical sensor (3).

11. The device of claim 7, wherein said anode comprises a central electrode and said cathode is a cylinder forming a coaxial needle-cylinder geometry.

12. The device of claim 1, wherein said optical sensor comprises at least one microscope.

13. The device of claim 1, wherein said optical sensor comprises at least one photographic camera.

14. The device of claim 5, wherein said image storage and processing system (4) comprises: a computer connected to said optical sensor (3), which receives, stores and analyzes, by artificial intelligence, the images of plasma produced by the samples when passing through the electric arc between the electrodes.

15. The device of claim 1, wherein it also comprises a PC USB connection; a lithium-ion battery; an integrated touch screen; the necessary elements to establish a wireless connection.

16. The device of claim 1, wherein said health disorder is a viral infection.

17. The device of claim 1, wherein said health disorder is a viral infection of COVID-19.

18. The device of claim 1, wherein said health disorder is a viral infection of pneumonia.

19. The device of claim 1, wherein said health disorder is diabetes.

20. The device of claim 1, wherein said health disorder is a bacterial infection.

21. The device of claim 1, wherein said health disorder is a bacterial infection of pneumonia.

22. The device of claim 1, wherein said health disorder is kidney failure.

23. The device of claim 1, wherein said health disorder is selected from the group consisting of: breast cancer, prostate cancer, lung cancer and colon cancer.

24. The device of claim 1, wherein said health disorder is alcohol present in the blood.

25. The device of claim 1, wherein said health disorder is related with the presence of cannabinoids in the blood.

26. The device of claim 1, wherein said health disorder comprises any disorder that can manifest itself through biomarkers present in exhaled breath.

27. The device for detecting diseases from breath samples of claim 1, comprising an electric discharge induced plasma digital spectrometer.

28. The device of claim 1, wherein said biological samples comprise urine samples.

29. The device of claim 1, wherein said biological samples comprise stool samples.

30. The device of claim 1, wherein it further comprises a sample reservoir wherein solid or liquid samples are introduced.

31. The device of claim 30, wherein said solid or liquid samples comprising stool and urine.

32. The device of claim 30, wherein it comprises means to vaporize liquid or solid samples.

33. The device of claim 32, wherein said means to vaporize said liquid or solid samples comprises a Laser.

34. A process for detecting health disorders from breath samples that uses the device of claim 1 and comprises the following steps:
a) providing a container with a breath sample;
b) providing a carrier gas that mixes with the breath sample to carry said sample into an ionization chamber in a homogeneous and controlled manner;
c) Ionizing said carrier gas and said breath by means of an electric arc;
d) capturing and storing images of the plasma generated in said electric arc;
e) evacuating the ionization chamber by circulating said pure carrier gas, in absence of a breath sample;
f) processing said images by artificial intelligence to determine if said images are compatible with breath samples from sick people;
g) giving a visual indication of the result.

35. A process for detecting compounds from gas samples that uses the device of claim 1 and comprises the following steps:
a. providing a carrier gas that mixes with the sample to carry said sample into an electric arc;
b. ionizing said carrier gas and said sample by means of an electric arc;
c. capturing and storing images of the plasma generated in said electric arc;

36. The process of claim 35 that also comprises the steps of:
a. processing said images by artificial intelligence to determine if said images are compatible with established parameters;
b. giving a visual indication of the result.

37. A container comprising flexible material evacuated and sterilized with only one gas entrance that could be filled with the exhales of breath as biological sample of the claim 1.

38. A process of image analysis called digital spectroscopy of the device of claim 1 comprising the following steps:

39. An image analysis process called digital spectroscopy that utilizes the device of claim 1 comprising the following steps:
a. generation of the database for the image training that represents the wavelengths to generate the spectra;
b. generation of the spectrum of each training image, fitting of the data and cross validation of the model;
c. generation of spectra of the samples to be analyzed and their prediction.
